(19) United States
(12) Patent Application Publication
(10) Pub. No.: US 2011/0144281 A1
(43) Pub. Date: Jun. 16, 2011

(54) AMPHIPHILIC POLYMERS HAVING A CHOLANE CORE

(75) Inventors: Xiao-Xia Zhu, Dollard-des-Ormeaux (CA); Juntao Luo, Sacramento, CA (US); Guillaume Giguere, Becancour (CA)

(73) Assignee: VALORISATION-RECHERCHE, SOCIETE EN COMMANDITE, Montreal, QC (CA)

(21) Appl. No.: 12/746,479

(22) PCT Filed: Dec. 5, 2008
(86) PCT No.: PCT/CA2008/002151

§ 371 Date: Mar. 2, 2011

Related U.S. Application Data
(60) Provisional application No. 60/992,543, filed on Dec. 5, 2007.

(51) Int. Cl. C08F 8/06; C07J 9/00; C07J 41/00; C07K 16/00; C07K 14/505; C07K 14/485; C07K 14/62; C07H 7/02; C07D 475/04

(52) U.S. Cl. 525/327.3; 552/553; 552/554; 530/391.1; 530/397; 530/399; 530/303; 536/1.11; 530/300; 544/261

(57) ABSTRACT

The present invention relates to a polymer comprising a cholane core having at least one derivatizable group covalently bonded thereto and a hydrophilic polymer chain covalently bonded to derivatizable group(s) and a process for producing it The present invention also relates to micellar aggregate formed from the polymer of the present.

### FIELD OF THE INVENTION

The present invention concerns polymers, and more particularly to amphiphilic polymers with a cholane core.

### BACKGROUND OF THE INVENTION

Compact structure and high segment density render star polymers with distinct physical properties in bulk, as a melt and in solution compared to their linear analogs. Star polymers with more than 3 arms, even up to 128 arms, have been studied from the synthetic and theoretical viewpoints (Inoue, K. Progress in Polymer Science 2000, 25, (4), 453-571; Hadjichristidis, N; Pitsikalis, M.; Pispas, S.; latrou, H. Chemical Reviews, 2001, 101, 3747-3792; Hirao, A.; Hayashi, M.; Tokuda, Y.; Haraguchi, N.; Higashihara, T.; Ryu, S. W. Polymer Journal 2002, 34, (9), 633-658; Vlassopoulos, D. Journal of Polymer Science Part B-Polymer Physics 2004, 42, (16), 2931-2941).

Poly(ethylene glycol) (PEG)-based star polymers have attracted much attention from researchers due to their well-known bioacceptability (Hawker, C. J.; Chu, F. K.; Pomery, P. J.; Hill, D. J. T. Macromolecules 1996, 29, (11), 3831-3838; Hou, S. J.; Taton, D.; Saule, M.; Logan, J.; Chaikof, E. L.; Gnanou, Y. Polymer 2003, 44, (18), 5067-5074; Lapienis, G.; Penczek, S. Macromolecules 2000, 33, (18), 6630-6632; Taton, D.; Saule, M.; Logan, J.; Duran, R.; Hou, S.; Chaikof, E. L.; Gnanou, Y. Journal of Polymer Science Part a-Polymer Chemistry 2003, 41, (11), 1669-1676). Branched PEG stars have shown to be more promising than linear PEG in certain biomedical and pharmaceutical applications. Much interest has been focused on asymmetric star polymers, where arms differ in molecular weight or chemical composition (Hirao, A.; Hayashi, M.; Tokuda, Y.; Haraguchi, N.; Higashihara, T.; Ryu, S. W. Polymer Journal 2002, 34, (9), 633-658; Beyer, F. L.; Gido, S. P.; Poulos, Y.; Avgeropoulos, A.; Hadjichristidis, N. Macromolecules 1997, 30, (8), 2373-2376; Tselikas, Y.; latrou, H.; Hadjichristidis, N.; Liang, K. S.; Mohanty, K.; Lohse, D. J. Journal of Chemical Physics 1996, 105, (6), 2456-2462). These star polymers are expected to exhibit interesting and unique physical performance owing to their branched asymmetrical architectures as well as hetero-phase structures.

Bile acids are surfactants biosynthesized in the liver of mammals as emulsifiers in the digestion of fats. Cholic acid, a major primary bile acid, possesses a rigid steroid skeleton structure and four hydrophilic groups located on one side of its rigid skeleton: three hydroxyl groups (all in α-position) and a carboxylic acid group. Several groups have synthesized polymers with either cholesterol cores or cholane cores, but due to solubility problems and incomplete polymerization, especially when all of the available OH groups are deprononated, the resultant polymers have been limited to a single hydrophilic chain (Kim et al. Langmuir, 2000, 16, 4792-4797; Han et al. Bioconjugate Chemistry, 2001, 12, 337-345; Koyama et al. Journal of Controlled Release, 2001, 77, 357-364). Thus, to date, amphiphillic polymers which have cholane cores, such as bile acids, in which all of the available derivatizable groups have been covalently bonded to hydrophilic polymer chains, have eluded synthesis.

### SUMMARY OF THE INVENTION

We have discovered a novel class of amphiphilic polymers which have a cholane core structure with one or more hydrophilic polymer chains attached to the core. Furthermore, we have discovered that in an aprotic solvent, such as dimethylsulfoxide, the polymers can be produced by attaching the hydrophilic polymer chains by a “core first” method by partially deprotonating the cholane core before addition of the hydrophilic polymer chains. This significantly reduces or essentially eliminates the solubility problems that have prevented the successful synthesis of such polymers. In one example, a new polymer (CA-PEG₄) has a core structure of cholic acid onto which four PEG chains are attached by the core-first and graft-from method. The cholane core imparts a spatial asymmetric distribution of the PEG chains, which are located on one side of the cholane backbone. Therefore, the polymer retains the hydrophobicity of cholic acid steroid skeleton on one face, while PEG chains modify its hydrophilicity on the other. Consequently, the amphiphilic asymmetric PEG stars can self-assemble into aggregates. Star polymers derived from cholic acid with poly(allyl glycidyl ether) arms have also been prepared similarly with well-defined molecular weight and low polydispersity. The double bonds on the polymer are used to introduce either amino groups or carboxylic acid groups to obtain amphiphilic polymers with cationic and anionic groups, respectively. The critical aggregation concentration in water changed with the arm length of the star polymer. A simple acetylation rendered the polymers thermosensitive, giving a cloud point ranging from 16 to 53° C. according to the degree of acetylation of the amino groups.

In one aspect of the present invention, there is provided a polymer comprising:
a) a cholane core having at least one derivatizable group covalently bonded to the core; and
b) a hydrophilic polymer chain covalently bonded to the at least one derivatizable group.

In another aspect of the present invention, there is provided a composition comprising:
a) a polymer comprising:
   i. a cholane core having at least one derivatizable group covalently bonded to the core; and
   ii. a hydrophilic polymer chain covalently bonded to the at least one derivatizable group.

In another aspect of the present invention, there is provided a micellar aggregate, the aggregate comprising:
a) in an aqueous solution, a plurality of polymers, each of the polymers comprising:
   i. a cholane core having at least one derivatizable group covalently bonded to the core; and
   ii. a hydrophilic polymer chain covalently bonded to at least one derivatizable group.

In one aspect of the present invention, there is provided a polymer comprising:
a) a bile acid core having at least one derivatizable group covalently bonded to the core; and
b) a hydrophilic polymer chain covalently bonded to at least one derivatizable group.

In another aspect of the present invention, there is provided a composition comprising:
a) a polymer comprising:
   i. a bile acid core having at least one derivatizable group covalently bonded to the core; and
   ii. a hydrophilic polymer chain covalently bonded to at least one derivatizable group.

In another aspect of the present invention, there is provided a micellar aggregate, the aggregate comprising:
b) in an aqueous solution, a plurality of polymers, each of the polymers comprising:
   i. a bile acid core having at least one derivatizable group covalently bonded to the core; and
   ii. a hydrophilic polymer chain covalently bonded to at least one derivatizable group.

In another aspect of the present invention, there is provided an amphiphilic polymer, the polymer comprising:
a) cholic acid; and
b) four PEG chains covalently bonded to the cholic acid.

In another aspect of the present invention, there is provided an amphiphilic polymer, the polymer comprising:
a) a cholane core having at least one derivatizable group covalently bonded to the core; and
b) a hydrophilic polymer chain covalently bonded to the at least one derivatizable group, the chain length of hydrophilic polymer being tunable to balance the amphiphilicity of the polymer.

There is further provided in the present invention a polymer comprising:
a) a cholane core having between one and four derivatizable group covalently bonded thereto;
b) a first monomer chain bonded to the derivatizable group, wherein the first monomer chain may optionally include a first functional group adapted to be chemically modified; and
c) a second functional group located at the end of the first monomer chain.

The first monomer chain may comprise a large number of units, but preferably between 1 and 200 units.

The polymer of the present invention may further comprise a second monomer chain bonded to the first monomer chain, wherein the second monomer chain may optionally include a functional group adapted to be chemically modified.

The second monomer chain may comprise between 1 to 200 units.

Alternatively, the polymer of the present invention may further comprise a second monomer chain comprising at least one unit bonded on each unit of the first monomer chain, the at least one unit of the second monomer optionally including a functional group adapted to be chemically modified.

In the above polymer, the second functional group may be further modified, if necessary, to add additional monomer units. Also contemplated that the second functional group being selected from the group consisting of, but not limited to, transferrin, asialoglycoprotein, antibodies, antibody fragments, low density lipoproteins, interleukins, GM-CSF, G-CSF, M-CSF, stem cell factors, erythropoietin, epidermal growth factor (EGF), insulin, asialoorosomucoid, mannose-6-phosphate, mannose, LewisX and sialyl LewisX, N-acetyllactosamine, galactose, lactose, thrombomodulin, fusogenic agents, polymixin B, hemagglutinin HA2, lysomotrophic agents, peptide, folic acid, and nucleus localization signals (NLS)

Typical functional groups include OH, NH₂, SH, CO₂H, amino acids, phosphates and the like and are located at the end of the chain and which may be further modified to attach peptides, proteins, nucleotides, glycopeptides, oligoglycerides, drug molecules, and the like.

In one example, the first and second monomer chain may form either block polymers or random copolymers. The derivatizable groups X covalently bonds the hydrophilic polymers to the cholane core. Typically, the derivatizable groups X include OH, NH₂, SH, CO₂H and the like, which when derivatized can form ethers, secondary or tertiary amines, thioethers, and esters or ketones, and the like.

Examples of the optional first functional group of one or both of the first and second monomer chain include, but not are not limited to, methyl, ethyl, halomethyl, haloethyl, allyl, vinyl, protected hydroxymethyl and hydroxyethyl, protected aminomethyl, aminoethyl, etc.), which themselves may be optionally further modified chemically; The functional groups on M or P units can be further extended to form branched polymers; M and P may form block or random copolymers;

The first monomer chain can be a compound selected from ethylene glycol or —CH₂—CH₂—O—, for example. The second monomer chain can be of the formula —CH₂—CHR—O—, wherein R is H, CH₃, CH₂CH₃, CH₂Cl, CH₂SH, CH₂CH₂SH, CH₂OH, CH₂CH₂OH, CH₂OCH₂CH═CH₂, for example.

In another aspect of the present invention, there is provided a process for preparing an amphiphilic polymer, the process comprising:
a) in an aprotic solvent, partially deprotonating a derivatizable group of a cholane core to produce a deprotonated group; and
b) condensing the deprotonated group with a hydrophilic polymer chain.

In another aspect of the present invention, there is provided a process for preparing CA-PEG₄, the process comprising:
a) in an aprotic solvent, deprotonating one of the available hydroxyl groups of cholic acid to produce an alkoxide group; and
b) condensing the alkoxide group with a hydrophilic polymer chain.

### BRIEF DESCRIPTION OF THE DRAWINGS

Further aspects and advantages of the present invention will become better understood with reference to the description in association with the following Figures, wherein:

FIG. 1 illustrates a general synthetic procedure for the preparation of CA-PEG₄ polymers.

FIG. 2 illustrates SEC traces of CA-PEG₄ star polymers (eluent: THF, RI detector, 1 mL/min). Sample details are given in Table 1;

FIG. 3 illustrates MALDI-TOF MS spectra of CA-PEG₄ star polymers I, II and III obtained using a N₂ laser at 337 nm wavelength with a 20 kV extraction voltage. Dithranol was used as the matrix in the presence of LiCl.;

FIG. 4 illustrates ¹H NMR spectra of a CA-PEG₄ star polymer (sample IV, n=30) in CDCl₃ before and after trifluoroacetylation;

FIG. 5 illustrates DSC traces of CA-PEG₄ star polymers obtained with a heating rate of 10° C./min (second heating curves);

FIG. 6 illustrates a comparison of melting points between linear PEG (▪) and CA-PEG₄ star polymers (▴). The Tₘ values of PEG stars are those obtained from DSC analysis and the data of linear PEGs are from literature (Hay, J. N.; Sabir, M.; Steven, R. L. T. Polymer 1969, 10, (3), 187-202; Beech, D. R.; Booth, C.; Dodgson, D. V.; Sharpe, R. R.; Waring, J. R. S. Polymer 1972, 13, (2), 73-7; Beech, D. R.; Booth, C.; Pickles, C. J.; Sharpe, R. R.; Waring, J. R. S. Polymer 1972, 13, (6), 246-8);

FIG. 7 illustrates the variation of the surface tension (ST) of selected CA-PEG₄ stars (sample I (left) and sample IV (right)) as a function of the molal concentration of the polymers;

FIG. 8 illustrates TEM images of the aggregates formed by sodium cholate at 0.040 molal (A) and CA-PEG₄ I at 0.025 molal (B) and III at 0.020 molal (C); and

FIG. 9 illustrates the preparation of the positively and negatively charged, and acetylated polymers from cholic acid;

FIG. 10 illustrates CPs measured for CA-OH(AGE₅-NH₂—NHCOOCH₃)₄ at degrees of acetylation ranging from 5% to 60% (bottom graph). The solutions were prepared at 0.1 wt % and heated at 0.1° C./min and scanned at a wavelength of 500 nm. The top graph is an example of thermogram for a sample with 10% acetylation.

### DETAILED DESCRIPTION OF THE INVENTION

### Definitions

Unless otherwise specified, the following definitions apply:

The singular forms “a”, “an” and “the” include corresponding plural references unless the context clearly dictates otherwise.

As used herein, the term “comprising” is intended to mean that the list of elements following the word “comprising” are required or mandatory but that other elements are optional and may or may not be present.

As used herein, the term “consisting of” is intended to mean including and limited to whatever follows the phrase “consisting of”. Thus the phrase “consisting of” indicates that the listed elements are required or mandatory and that no other elements may be present.

As used herein, the term “cholane” is intended to mean a class of steroid compounds which is characterized as having a hydrocarbon skeleton with four fused rings, generally arranged in a 6-6-6-5 members on the cycles. One example of a cholane includes, but is not limited to, cholic acid, which is a bile acid.

As used herein, the term “bile acid” is intended to mean a steroid structure with four fused rings, a five or eight carbon side chain terminating in a carboxylic acid group. Examples of bile acids include, but are not limited to, cholic acid, chenodeoxycholic acid, deoxycholic acid, lithocholic acid and their derivatives such as glycocholic acid and taurocholic acid.

As used herein, the term “hydrophilic polymer” is intended to mean repeating units of epoxy compounds based on an ethylene oxide structure. The polymer can have a plurality of units, preferably up to 1000 units and more preferably 80 units. Examples of such hydrophilic polymers include, but are not limited to, poly(ethylene glycol) (PEG) and poly(allyl glycidyl ether). One skilled in the art will readily recognize that many hydrophilic polymers are available and may be used to practice the present invention.

As used herein, the term “derivatizable group” is intended to mean a chemical functional group which may be reacted to another activated species to form a covalent bond between the species and the group. Examples of derivatizable groups include, but are not limited to, OH, SH, NH₂, CO₂H, and the like.

As used herein, the term “partially deprotonated” is intended to mean that at least one of several available derivatizable groups is deprotonated under conditions in which the deprotonated species remains soluble in the aprotic solvent used. Broadly speaking, between 5 and 99% deprotonation of the total number of derivatizable groups is desirable. In one example provided in the instant invention, partial depronation is 25% which means that one of the four OH groups of the cholic acid core was deprotonated to maintain solubility of the polymer. To ensure the success of the anionic polymerization: the deprotonated species should remain soluble in the aprotic solvent. In the example described herein, the aprotic solvent is DMSO. Furthermore, the degree of deprotonation has to be sufficient for the anionic polymerization to take place.

Broadly speaking, the present invention concerns amphiphilic polymers which have a cholane core having at least one derivatizable group covalently bonded to the core. The derivatizable groups can be OH, SH, NH₂ or CO₂H groups. In one example described herein, the derivatizable groups are OH groups. The invention also contemplates cores in which mixed derivatizable groups are covalently bonded to the fused ring system of the cholanes. Generally speaking, one or more derivatizable groups may be present. In one example described herein, four derivatizable groups are covalently bonded to the fused ring system of the cholane core. One or more of the derivatizable groups can be covalently bonded to a hydrophilic polymer chain. In the example described herein, four

PEG chains are covalently bonded to the respective deriavtizable groups. The chain length of the hydrophilic polymers are tunable to balance the amphiphilicity of the polymer.

Also contemplated by the present invention are compositions of the amphililic polymers. The compositions may further include carriers, fillers, excipients, and the like.

There is further provided in the present invention a polymer comprising:

a) a cholane core having between one and four derivatizable group covalently bonded thereto;

b) a first monomer chain bonded to the derivatizable group, wherein the first monomer chain may optionally include a first functional group adapted to be chemically modified; and

c) a second functional group located at the end of the first monomer chain.

The first monomer chain may comprise a large number of units, but preferably between 1 and 200 units.

The polymer of the present invention may further comprise a second monomer chain bonded to the first monomer chain, wherein the second monomer chain may optionally include a functional group adapted to be chemically modified.

The second monomer chain may comprise between 1 to 200 units.

Alternatively, the polymer of the present invention may further comprise a second monomer chain comprising at least one unit bonded on each unit of the first monomer chain, the at least one unit of the second monomer optionally including a functional group adapted to be chemically modified.

In the above polymer, the second functional group may be further modified, if necessary, to add additional monomer units. Also contemplated that the second functional group being selected from the group consisting of, but not limited to, transferrin, asialoglycoprotein, antibodies, antibody fragments, low density lipoproteins, interleukins, GM-CSF, G-CSF, M-CSF, stem cell factors, erythropoietin, epidermal growth factor (EGF), insulin, asialoorosomucoid, mannose-6-phosphate, mannose, LewisX and sialyl LewisX, N-acetyllactosamine, galactose, lactose, thrombomodulin, fusogenic agents, polymixin B, hemagglutinin HA2, lysomotrophic agents, peptide, folic acid, and nucleus localization signals (NLS)

Typical functional groups include OH, NH₂, SH, CO₂H, amino acids, phosphates and the like and are located at the end of the chain and which may be further modified to attach peptides, proteins, nucleotides, glycopeptides, oligoglycerides, drug molecules, and the like.

In one example, the first and second monomer chain may form either block polymers or random copolymers. The derivatizable groups X covalently bonds the hydrophilic polymers to the cholane core. Typically, the derivatizable groups X include OH, NH₂, SH, CO₂H and the like, which when derivatized can form ethers, secondary or tertiary amines, thioethers, and esters or ketones, and the like.

Examples of the optional first functional group of one or both of the first and second monomer chain include, but not are not limited to, methyl, ethyl, halomethyl, haloethyl, allyl, vinyl, protected hydroxymethyl and hydroxyethyl, protected aminomethyl, aminoethyl, etc.), which themselves may be optionally further modified chemically; The functional groups on M or P units can be further extended to form branched polymers; M and P may form block or random copolymers;

The first monomer chain can be a compound selected from ethylene glycol or —CH₂—CH₂—O—, for example. The second monomer chain can be of the formula —CH₂—CHR—O—, wherein R is H, CH₃, CH₂CH₃, CH₂Cl, CH₂SH, CH₂CH₂SH, CH₂OH, CH₂CH₂OH, CH₂OCH₂CH═CH₂, for example.

Specifically, the present invention concerns amphiphilic polymers, which may be asymmetric or may be so-called “star” polymers, and which are produced by the grafting of PEG chains of different lengths on the cholane core of cholic acid. We have achieved this by anionic polymerization of ethylene oxide, which provides polymers with very low polydispersity. It is easy to apply the same grafting method to other bile acids or compounds with multiple derivatizable functional groups of this kind. The PEGylated cholic acid derivatives can form spherical micellar aggregates in water, providing interesting reservoir for hydrophobic compounds that may be explored for use as drug delivery vehicles. The OH groups of the PEG chains may be further modified to introduce other functional groups for different applications. Further experiments will be carried out to study the formation of mixed micelles. Contemplated uses for the micelles or mixed micelles include, but are not limited to, drug carriers or cosmetics. Micelles can be prepared easily by dissolving the synthesized polymers in water above their critical micellar concentrations (CAC). Mixed micelles can be prepared by dissolving the amphiphilic polymers together with bile acids, fatty acids or other similar or different polymeric or oligomeric derivatives of bile acids. Active ingredient(s), particularly agents that are hydrophobic or amphiphilic can be incorporated into these micelles. The active ingredient can be release over time along with the disruption or solublization of the micelles.

Poly(ethylene glycol) (PEG) arms are grafted onto a cholane core via anionic polymerization to obtain asymmetric star-shaped polymers. The anionic polymerization of ethylene oxide was optimized in different solvents and with different degrees of deprotonation of the initiating hydroxyl groups on a cholic acid derivative. In dimethylsulfoxide, 25% deprotonation of the hydroxyl groups on the cholane core afforded a better control over the molar mass and polydispersity of the polymer obtained. Well-defined cholic acid-PEG stars (polydispersity index ca. 1.05) with tunable molar masses (ca. 1000-13000) were obtained and characterized by the use of size exclusion chromatography, MALDI-TOF mass spectrometry, NMR spectroscopy and thermal analysis. The critical aggregation concentrations of the star polymers were determined and spherical aggregates of the polymers with different PEG chain lengths were observed by transmission electron microscopy using the freeze-fracture etching technique and compared with results obtained from dynamic light scattering measurements.

### Synthesis and Methodology

A general method for the synthesis of the polymers of the present invention is shown below and is disclosed merely for the purpose of illustration and are not meant to be interpreted as limiting the processes to make the polymers by any other methods.

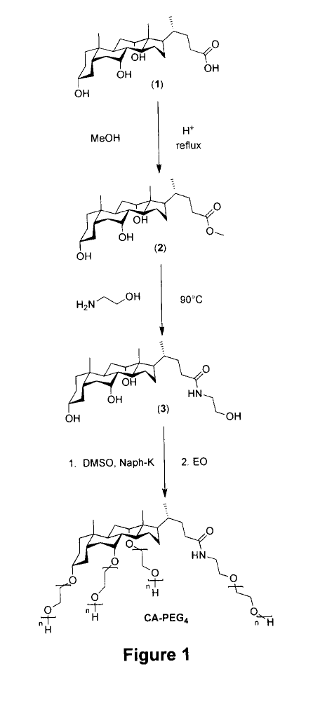

FIG. 1 illustrates a general synthetic procedure for the preparation of CA-PEG₄ polymers of the present invention:

### EXAMPLES

### Materials

Cholic acid (98%) and 2-aminoethanol (98%) were purchased from Aldrich and used without further purification. Dimethylsulfoxide (DMSO, from Aldrich) was dried by refluxing with calcium hydride for 48 h before distillation. Tetrahydrofuran (THF) was dried with sodium in the presence of benzophenone and was distilled after the solution turned dark blue. Potassium naphthalene was prepared directly in dry THF from naphthalene (>99%, Aldrich) and potassium (98% in mineral oil, Aldrich) with a concentration of 0.45 mol/L (titrated with a standard hydrochloric acid solution). Ethylene oxide (EO) was distilled from a trap with a 1.6 mol/L n-butyl lithium solution in hexane (from Aldrich) to another trap after passing through a calcium hydride drying column. All glassware used in the anionic polymerization was flame-dried under vacuum before use.

### 1. Preparation of (2′-hydroxylethylene)-3α,7α,12α-trihydroxy-5β-cholanoamide (3)

Cholic acid methyl ester 2 (8.0 g), prepared from cholic acid via a previously published procedure (Benrebouh, A.; Zhang, Y. H.; Zhu, X. X. Macromolecular Rapid Communications 2000, 21, (10), 685-690), was dissolved in 50 mL of dry 2-aminoethanol and refluxed for 4 h. The reaction solution was then cooled and 50 mL of ice water was poured into the solution. The product was precipitated and filtered at room temperature, then dissolved in hot methanol followed by the addition of ethyl acetate (4 times excess) to precipitate again. After filtration and drying in an vacuum oven, 8.0 g of product (3) was obtained with a yield of 93%. Elemental analysis: C₂₆H₄₅NO₆, calculated: C, 69.14%; H, 10.04%; N, 3.10%. found: C, 69.15%; H, 10.49%; N, 3.14%. FTIR (cm⁻¹): 1655 (sharp), 3288 (sharp). ¹H NMR (400 MHz, DMSO-d₆, ppm): δ=0.58 (3H, s, 18-CH₃), 0.81 (3H, s, 19-CH₃), 0.92 (3H, d, J=6.4 Hz, 21-CH₃), 1.0-2.2 (m, protons on the steroidal skeleton backbone), 3.08 (2H, q, J. 6.1 Hz, 25-NCH₂), 3.18 (1H, m, 3-CH), 3.35 (2H, q, J. 5.7 Hz, 26-CH₂O), 3.60 (1H, m, 7-CH), 3.77 (1H, m, 12-CH), 4.01 (1H, d, J=3.3 Hz, 12-COH), 4.10 (1H, d, J=3.5 Hz, 7-COH), 4.32 (1H, d, J=4.3 Hz, 3-COH), 4.62 (1H, t, J. 5.4 Hz, 26-COH), 7.75 (1H, t, J=5.7 Hz, 24-CONH). ¹³C NMR (100 MHz, DMSO-d₆, ppm): δ=13.21, 17.99, 23.49, 23.67, 27.08, 28.16, 29.43, 31.26, 32.58, 33.36, 35.25, 35.26, 36.07, 36.18, 42.23, 42.26, 42.38, 46.59, 46.99, 60.84, 67.11, 71.30, 71.88, 173.65. LCMS: t_{R} 10.36 min, m/z 452 M+H⁺.

### Preparation of 50-Cholane-3α,7α,12α,24-tetrol

Alternatively, the carboxylic acid group of bile acids can be reduced to the alcohol, among several methods, by the method used by Kihira et al. (Kihira, K.; Mikami, T.; Ikawa, S.; Okamoto, A.; Yoshii, M.; Miki, S.; Mosbach, E. H.; Hoshita, T. Steroids 1992, 57, 193-198). In a typical reaction, 10 g of CA (24.5 mmol) were dissolved in 400 mL of dry THF under nitrogen atmosphere with 13 mL (93.9 mmol) of triethylamine. To this solution, 8 mL (83.7 mmol) of ethylchloroformate were added dropwise and the solution left to react at room temperature for 2 hours. Then, 10.67 g of sodium borohydride dissolved in 11 mL of water were slowly added and reacted for 3 hours. After adding 200 mL of water and concentrated hydrochloric acid to neutralize the solution, the aqueous phase was extracted 3 times with ethyl acetate. The organic phase was dried over magnesium sulfate, filtered and evaporated. The solid residue was recrystallized in methanol and the resulting crystal dried in a vacuum oven. Yield: >96% of tetranol of CA.

### 2. Anionic Polymerization of Ethylene Oxide

In a typical procedure, all the glassware and needles were flame-dried under vacuum and purged with argon 3 times. 0.45 g of 3 (1 mmol) was placed into a 100 mL flask, charged with 40 mL of dry DMSO. The potassium naphthalene solution in THF (0.43 mol/L) was introduced (9.2 mL, 1 equiv., or 2.3 mL, 0.25 equiv.) dropwise into the vigorously stirred flask via a canula by high pressure argon. Then, dry ethylene oxide chilled in dry ice/acetone, was introduced into the flask and polymerized at 40° C. for 48 h. The reaction mixture was neutralized and quenched with concentrated HCl. The DMSO solution was extracted with hexane (50 mL×3) to remove naphthalene and DMSO was removed by distillation under vacuum. A small amount of THF was added to the residues to dissolve the polymer and to precipitate the salt. After filtering off the salt, the polymer was concentrated to dryness by rotary evaporation and high vacuum. The polymer was characterized by the use of various techniques as described below.

### Preparation of CA-24OH(AGEₓ)₄

In a typical procedure, all glassware was flame-dried under vacuum and purge with argon three times. In a dry flask, 1 g of CA-OH24 (2.5 mmol) was placed in a 250 mL Schlenk flask and purged with argon. The flask was then charged with 67 mL of freshly distilled DMSO. A 6.3 mL (0.4 M, 2.5 mmol) of potassium naphthalene solution was added stepwise to the stirred solution of DMSO maintained at 30° C. To obtain 5 repeating-unit polymer chain length, 6.0 mL (5.78 g; 51 mmol) of freshly distilled allyl glycidyl ether (AGE) were transferred to the flask. The polymerization was let to proceed overnight and then quenched with concentrated hydrochloric acid and extracted with 3 portions (30 mL) of hexanes. Water (200 mL) was added to DMSO and extractions with dichloromethane (3×100 mL) were performed. The solvent was evaporated and the sample was dried under high vacuum. Yield: >90%.

Functional groups such as COOH or NH₂ can be introduced as pendant groups of the polymer chains and examples of the procedures are described below:

The addition of carboxylic acid pendant groups was achieved by the addition of 3-mercaptopropionic acid on the CA-24OH(AGEₙ)₄ was performed by dissolving first the polymer in THF in a ratio of 2.75 mL for 1 g of polymer. Then, 5 and 0.15 eq., according to the number of double bonds, of the 3-mercaptopropionic acid and AIBN were added, respectively, and the solution was refluxed for 5 hours. The most volatile compounds were removed with a rotary evaporator and a vacuum distillation was performed to remove the less volatile ones. The viscous liquid obtained from this distillation was purified by dialysis.

Yield: >94%.

The addition of amino Pendant Groups was carried out as the following: CA-24OH(AGEₓ)₄ was dissolved in methanol ( 1/20, w/v), 5 equivalents of cysteamine hydrochloride and 0.5 equivalent of AIBN per double bond were added. The solution was refluxed for 36 hours. Methanol was evaporated and the residual syrup was dissolved in chloroform. Sodium hydroxide pellets were added to neutralize the ammonium chloride salt and the organic layer was washed with water. After solvent evaporation, the resulting viscous liquid was dialyzed against deionized milli-Q water. Yield>95%.

### 3. Characterization Techniques

The infrared spectra were recorded on the Bomen MB-100 Fourier transform IR spectrometer at room temperature, potassium bromide pellets were made with ground polymer. ¹H NMR spectra of polymers and cholic acid derivatives were recorded on a Bruker AMX400 spectrometer operating at 400 MHz for protons. The molar mass of the polymers were determined by size exclusion chromatography (SEC) in THF at 25° C. with a flow rate of 1 mL/min in reference to linear PEG standards. The thermal transitions of the polymers were analyzed on a TA DSC2910 differential scanning calorimeter (DSC).

MALDI-TOF mass spectrometry was performed on a Bruker Autoflex MALDI-TOF mass spectrometer, which used a 20 kV extraction voltage and a N₂ laser of 337 nm wavelength. Dithranol (1,8-dihydroxy-910H-anthralenone) (Sigma) was used as a matrix with the addition of LiCl for the MALDI-TOF MS analysis. A peptide calibration standard with a molecular weight range of 1-4 kDa and a protein calibration standard with a molecular weight range of 3-25 kDa were used to calibrate the molar masses of the star polymers.

Liquid chromatography ultra-violet conditions: Betasil C₁₈ column 150×4.6 mm, A=5% v/v trifluoroacetic acid (TFA) in H₂O and B=5% v/v TFA in CH₃CN, gradient of B from 20% to 80% over 20 min, flow rate at 0.5 mL/min., injection volume: 10 μL. MS conditions: scan 100-800, cone voltage 30 kV, temperature 400° C., mode (polarity) positive. Surface tension (ST) measurements were performed on a First Ten Angstroms instrument model FTÅ200 with milli-Q water. The pendant droplet method was used to calculate the critical aggregation concentration (CAC) of the polymers. The instrument was calibrated using the needle width as reference.

The average size of the aggregates was measured by dynamic light scattering (DLS) on a Brookhaven Instrument (model BI-200SM) equipped with a 532 nm laser. For the size distribution of the different samples in solution, the inverse Laplace transform was performed with MatLab using the regularization function. The average hydrodynamic radius (Rₕ) was calculated according to the apparent F of the Williams-Watts function.

For transmission electron microscopy (TEM), different sample concentrations were prepared in milli-Q water for freeze-fracture. A small amount of these solutions were dropped onto a good sample carrier, and then frozen in liquid propane. The frozen samples were then mounted on the sample holder of a BAL-TEC freeze etching instrument (model BAF060). Samples were then fractured, let sublimate for less than 30 seconds before the newly created surface was coated from an angle of 45° (shadowed) with 2 nm of platinum-doped carbon. A 10 nm layer of carbon was then applied perpendicularly. The samples were placed in distilled water to make the platinum-carbon replica float on the water surface. They were then deposited on carbon-coated copper grids. The replicas were examined on a JEOL JEM-2000FX TEM operating at an acceleration voltage of 80 kV.

### Preparation of the Core Precursor Compound 3

Cholic acid methyl ester was aminolyzed with 2-aminoethanol (Scheme 1). The FTIR spectrum of 3 shows very sharp peaks at 1655 cm⁻¹ (C=0), 3288 cm⁻¹ (NH) and 1570 cm⁻¹ (NH) typical of an amide and no peaks attributable to an ester (1736 cm⁻¹ for 2). This indicates the complete conversion of the methyl ester reacts to the corresponding amide.

¹H NMR and ¹³C NMR spectra also confirm the structure of the amide 3.

### Optimization of Polymerization Conditions

After deprotonation with potassium naphthalene, the solubility of the alkoxide becomes even lower due to the tendency of the alkoxide to aggregate; the reaction solution turned milky and opaque. DMSO was used as a solvent to increase the solubility of the salted precursor 3 in polymerization. It was observed that the phase separation occurred when the monomer was introduced less than 40 equiv. in order to generate short PEG chains, and polymers with wide PDI were obtained. A homogenous polymer solution was obtained when 80 equiv. of EO was charged into the reaction mixture and a narrow dispersed polymer was isolated (PDI: 1.07), even though the mixture still became milky at the beginning stage of polymerization with 100% deprotonation of precursor 3.

Although narrow disperse polymers can be obtained using a full deprotonation of the hydroxyl groups with large quantities of monomer added (usually more than 80 equiv.), phase separation are still encountered in the preparation of star polymers with short PEG chains. Star PEG polymers can be prepared with a partial deprotonation of alcohol groups due to the rapid proton exchange between the dormant hydroxyl groups and the active alkoxides. The proton NMR study of the prepared star-shaped polymer showed that four PEG chains were attached on one cholane core.

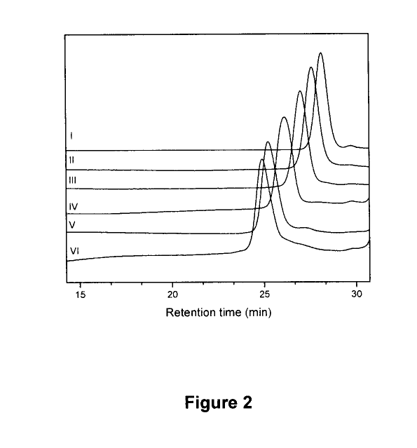

In order to avoid the milky transition, 25% deprotonation (Scheme 1) was employed in the in the preparation of samples I to VI. The polymerization solutions remained transparent throughout the entire duration of the polymerization. As shown in Table 1, a series of star polymers were prepared with narrow polydispersities (FIG. 2), and the average molecular weights were analyzed with SEC, MALDI-TOF MS and ¹H NMR.

TABLE 1
Properties of the CA-PEG₄ star polymers grafted with PEG chains of
different lengths.

| | EO | Mₙ (PDI) | | | | CAC |
|---|---|---|---|---|---|---|
| Sample | units | Theo. | SEC | MALDI-TOF | NMR | (millimolal) |
| I | 20 | 1330 | 1110 (1.04) | 1360 (1.03) | 1440 | 9 |
| II | 40 | 2210 | 1510 (1.04) | 1810 (1.03) | 2700 | 12 |
| III | 60 | 3090 | 2190 (1.04) | 2430 (1.03) | 3500 | 15 |
| IV | 120 | 5730 | 3500 (1.03) | 3640 (1.03) | 5200 | 16 |
| V | 200 | 9250 | 5870 (1.05) | 5980 (1.03) | 8830 | 19 |
| VI | 300 | 13650 | 7320 (1.03) | 6860 (1.02) | 10280 | 19 |

Note:
Mₙ: number-average molecular weight obtained by SEC relative to linear PEG standards, by MALDI-TOF MS with dithranol as a matrix and peptide standards, or by ¹H NMR peak integration of methyl proton signals on the cholic acid core and the methylene proton signals on the PEG chains, presented in comparison to the theoretical value calculated based on the amount of EO used;
CAC: the critical aggregation concentration obtained by surface tension measurement with an accuracy of ± 1 millimolal.

### Confirmation of the Structures of the CA-PEG₄ Star Polymers

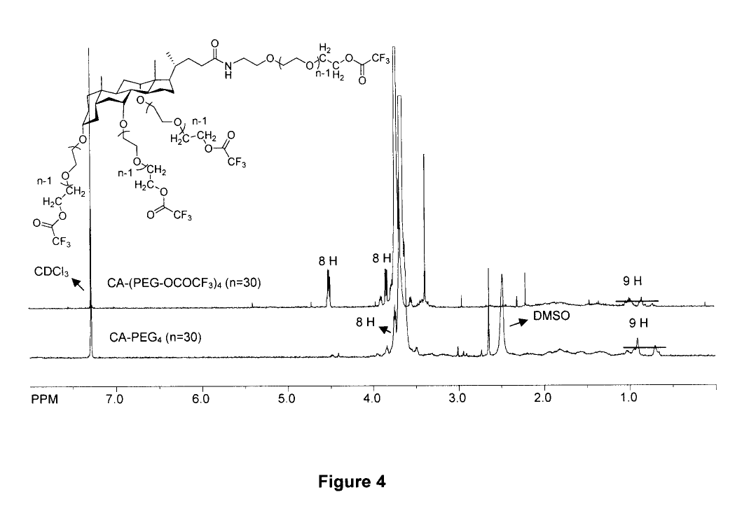

FTIR spectra show a decrease in the carbonyl band at 1641 cm⁻¹ with the increasing length of PEG chains grafted on the cholane core 3 from sample I to sample V (data not shown), indicating a qualitative chain growth. It is important, however, to ensure that all four hydroxyl groups on the cholane core are grafted with a PEG chain when 25% deprotonation of the hydroxyl groups were used. To do this, the CA-PEG₄ star polymers can be treated with the trifluoroacetic anhydride, followed by NMR analysis of the integral of the proton signal intensity of the CH₂ adjacent to the trifluoroacetyl group. FIG. 4 shows the NMR spectra of the CA-PEG₄ sample with a PEG-chain length of 30 units before and after the reaction. It is clear that the protons of CH₂ on the a-position adjacent to trifluoroacetate were shifted from 3.8 to 4.5 ppm, and the protons of the CH₂ on the β-position of trifluoroacetate were also shifted to a lower field. In the proton NMR spectra, the ratios of the integral of methylene protons at 4.5 ppm and 3.8 ppm to that of the three methyl groups on cholane core were all observed to be 8:9, indicative of four PEG chains on each cholane core structure.

### Analyses of the Molar Mass of the CA-PEG₄ Star Polymers

The theoretical values of molecular weight of the star polymers can be calculated based on the amounts of initiator and monomer (EO) added. However, SEC and MALDI-TOF MS analyses gave lower molecular weights than the theoretical values (Table 1). It is known the star-shaped polymers have a smaller hydrodynamic radius than the corresponding linear polymers with the same molecular weight. Therefore, when linear PEGs were used as the calibration standards, the molecular weights of star-shaped PEGs may be under-estimated in SEC analysis.

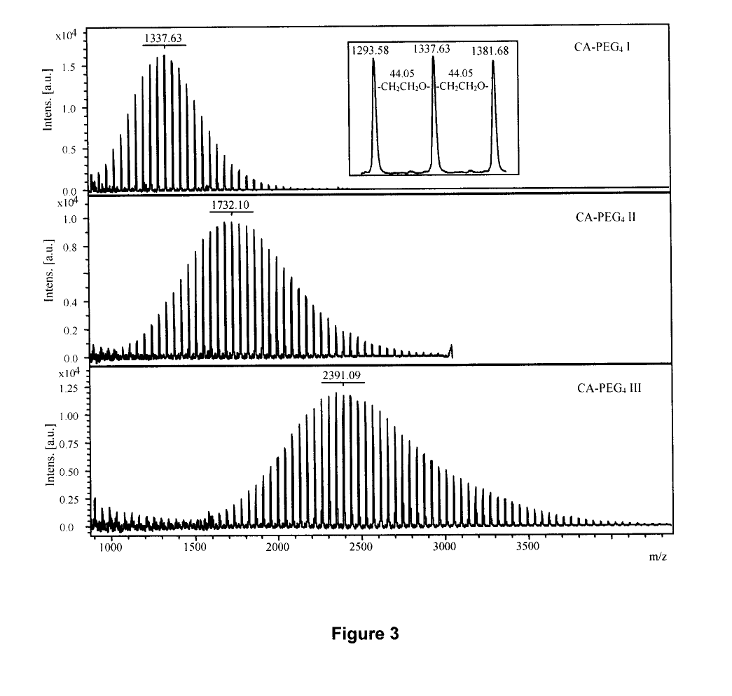

Both MALDI-TOF MS and NMR can be used for the accurate measurements of polymers of lower molar masses. The molar masses calculated from the ¹H NMR signals, using the ratio of the proton signals of PEG chains and the methyl group (position 18) on cholic acid, are closer to the theoretical molecular weights of the CA-PEG₄ stars than the SEC results. MALDI-TOF MS is particularly suitable in the analysis of polymers of low polydispersity of molar masses. For the CA-PEG₄ stars with lower molar masses (samples I to IV), high resolution spectra were obtained by MALDI-TOF (FIG. 3) showing symmetric distributions of the molecular masses. The Mₙ values obtained here are closer to the theoretical values for these samples in comparison to the values obtained by SEC. For star polymers with higher molecular weight (samples V and VI), the signal to noise ratio decreased in MALDI-TOF experiments. MALDI-TOF MS yielded lower molecular weight than the SEC experiments for the high molar mass polymers such as sample VI (Table 1 both SEC and MALDI-TOF MS methods gave similar values for the very low polydispersity of the star-shaped polymers (<1.05), which serves as a proof of a controlled anionic polymerization process.

### Thermal Analysis of CA-PEG₄ Star Polymers

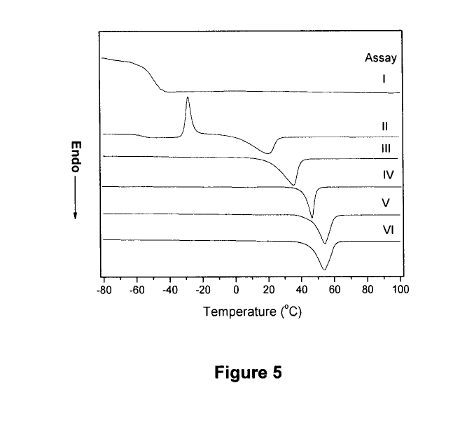

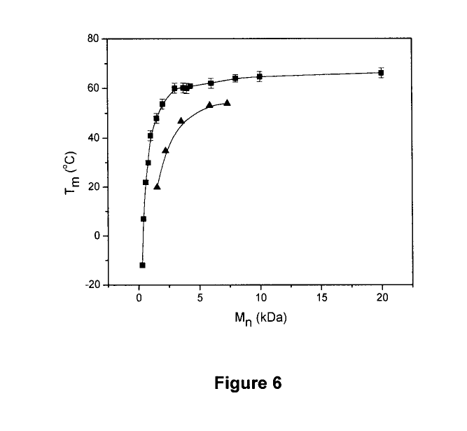

Melting point suppression is a well-known effect of the PEG chains in star polymers, because of the defective PEG crystal lattice caused by the core and by the lower molecular weight of the PEG chains (Chen, E. Q.; Lee, S. W.; Zhang, A.; Moon, B. S.; Honigfort, P. S.; Mann, I.; Lin, H. M.; Harris, F. W.; Cheng, S. Z. D.; Hsiao, B. S.; Yeh, F. Polymer 1999, 40, (16), 4543-4551; Chen, E. Q.; Lee, S. W.; Zhang, A. Q.; Moon, B. S.; Mann, I.; Harris, F. W.; Cheng, S. Z. D.; Hsiao, B. S.; Yeh, F. J.; von Merrewell, E.; Grubb, D. T. Macromolecules 1999, 32, (15), 4784-4793). The melting points of crystalline polymers may also depend on the thermal history of the sample. In order to erase the thermal history of the samples, the DSC thermograms of the CA-PEG₄ samples were recorded during the second heating at 10° C./min (FIG. 5). The CA-PEG₄ star polymer with very short PEG chains (i.e., sample I with 4 EO units on each chain) have a glass transition without crystallization and melting. The PEG chains here may be too short to form any crystalline domain. A star polymer with a Mₙ of 1510 (polymer II, average 6 EO units) shows a weak glass transition, a sharp exothermal crystallization peak and a broad melting point. From polymers III to VI, the increasing molar mass of the samples also raises the melting points. The melting points of the polymers seem to depend on the size of the crystalline domains, which may be larger with increasing length of the PEG chains. FIG. 6 shows that the melting points of the PEG stars are always lower than those of the linear PEGs of the same molar mass. This is an indication of the radial structure of the star PEGs with a higher packing density.

### The Aggregation of the CA-PEG₄ Star Polymers

### (1) Determination of the CAC

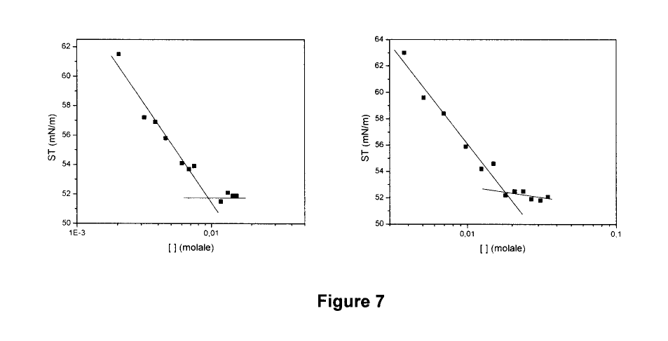

Because of the asymmetric structure and the amphiphilicity of the CA-PEG₄ star polymers, they easily aggregate in water. The aggregation of these polymers was then studied by the use of surface tension technique. Molal concentration (mol of solute per 1 kg of the solvent) was used because of its convenience in the calculation of concentration for samples of varying volume but of known weight. The value should be close to the molar concentration (mol of solute per liter of solution) at low concentrations. The surface tension of the solution should decrease with increasing concentration of surfactant molecules and becomes stable above a certain concentration of the surfactants, which can be defined as the CAC of the surfactant. While the hydrophobic parts of the surfactants are unchanged, the decrease in ST with the increasing hydrophilic segments in the surfactant is almost invariable. FIG. 7 indicates that the CACs of the star polymers are visible, indicating the formation of the aggregates. All the CA-PEG₄ star polymers prepared in this study have a CAC (Table 1), including the one with the longest PEG chains (sample VI, n=75), indicating that these polymers all maintain a reasonable balance of amphiphilicity to allow hydrophobic interactions of the convex faces of cholane core in the formation of stable aggregates above their CACs. The CACs of CA-PEG₄ are higher than the CAC of sodium cholate (8 millimolal) (Reis, S.; Moutinho, C. G.; Matos, C.; de Castro, B.; Gameiro, P.; Lima, J. Analytical Biochemistry 2004, 334, (1), 117-126), and increased from 9 to 19 millimolal with increasing PEG chain lengths due to the higher hydrophilicity of the longer PEG chains.

### (2) DLS and TEM Analysis CA-PEG₄ Star Polymers

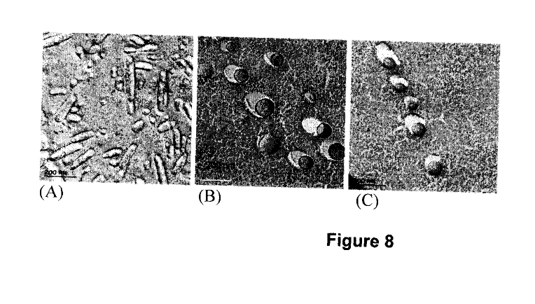

TEM images of the star polymers shown in FIG. 8 provide unequivocal evidence for the formation of micellar aggregates. Spherical aggregates with sizes around 100-130 nm are shown in the images obtained above the CACs of the CA-PEG₄ star polymers, which are significantly different from the cylindrical aggregates formed by sodium cholate.

TEM provided images of a limited number of the frozen micelles (not large enough to provide a statistical distribution of the size). DLS experiments can be used to study the size and distribution of the micelles in solution. Selected samples were studied and Rₕ is calculated according to the Stoke-Einstein equation assuming a spherical structure, which is not the case for the aggregates of sodium cholate, leading to a smaller hydrodynamic diameter than the average rod length. The large width of the distribution as shown by the β value can be explained by the stepwise aggregation of sodium cholate that unavoidably gives many species in solution. For samples I and III, the average hydrodynamic diameters are larger than those shown in the TEM images. The discrepancy is not too large and could be due to the more hydrated state of the micelles in solution.

Cytotoxicity Test: The MTT assay and the MTS assay are laboratory tests and standard colorimetric assays for measuring the activity of enzymes that reduce MTT or MTS+PMS to formazan, giving a purple color. This mostly happens in mitochondria, and so the assays are therefore largely a measure of mitochondrial activity. It can be used to determine the cytotoxicity of materials. Agents with cell toxicity result in mitochondrial dysfunction. Yellow MTT (3-(4,5-dimethylthiazol-2-yl)-2,5-diphenyltetrazolium bromide, a tetrazole) is reduced to purple formazan in the mitochondria of living cells. The absorbance of this colored solution is quantified by measuring at a wavelength between 500 and 600 nm on a spectrophotometer. The standard MTT assays were carried out with three CA-PEG₄ polymers (1850<Mₙ<15000). Cell viability remained 100% within experimental error for a concentration of the CA-PEG₄ polymers up to 0.1 mg/ml and 80% for a concentration of the CA-PEG₄ polymers at 10 mg/ml, indicating very low cytotoxicity of the polymers.

Loading of a drug. Ibuprofen has a very limited solubility in neutral water and remains as a crystalline residue when added in water, even after vigorous agitation. The addition of CA-PEG₄ (Mₙ=1850 and 2400) to the system increased the miscibility of the water-ibuprofen mixture. The gradual addition of CA-PEG₄ (above the expected CMC at ca. 15 mM) produced a cloudy emulsion and then a clearer solution, indicating the solubilisation of the drug ibuprofen in the micellar system.

### Preparation of CA-OH(AGEₙ)₄

5β-Cholane-3α,7α,12α,24-tetrol (CA-OH) was prepared from cholic acid according to a literature procedure (K. Kihira, T. Mikami, S. Ikawa, A. Okamoto, M. Yoshii, S. Miki, E. H. Mosbach and T. Hoshita, Steroids, 1992, 57, 193-198). We used anionic polymerization where the alcoholate groups of CA-OH form ether linkages upon ring opening of the oxirane derivatives. Allyl glycidyl ether (AGE) is used to allow the subsequent addition of thiolated compounds containing carboxylate or amino groups. Naphthalene radical anions were obtained by mixing of naphthalene and potassium in anhydrous tetrahydrofuran at an approximate concentration of 0.40 M and further titrated with a standard 0.1 M hydrochloric acid aqueous solution. AGE (≧99%) and dimethyl sulfoxide (DMSO, ≧99%) were obtained from Aldrich and dried over calcium hydride prior to reflux (2 hours) under reduced pressure followed by distillation.

In a typical procedure, all glassware was flame-dried under vacuum and purged with argon three times. In a dry flask, 1 g of CA-OH (2.5 mmol) was placed in a 250 mL Schlenk flask and purged with argon. The flask was then charged with 67 mL of freshly distilled DMSO with the aid of a double-ended needle using a positive pressure. Potassium naphthalene solution (6.3 mL, 2.5 mmol) was added stepwise, and after the disappearance of the green color, slowly to the stirred solution of DMSO maintained at 30° C. In order to obtain 5 repeat units in length for every initiating site on CA-OH, 6.0 mL (5.78 g, 51 mmol) of freshly distilled AGE were transferred to the flask in the same manner as DMSO. The polymerization was allowed to proceed overnight and finally quenched with concentrated hydrochloric acid and extracted with hexanes (3×30 mL). Water (200 mL) was added to the DMSO medium and extractions with dichloromethane (3×100 mL) were performed, followed by the removal of the solvent on a rotary evaporator. Final traces of solvent were removed under high vacuum. Yield: >90%. ¹H NMR (400 MHz, CDCl₃, ppm, n=5): spectrum shown in FIG. 2; 0.69 (18-CH₃), 0.89 (19-CH₃), 0.98 (21-CH₃), 3.55 (5H, protons on the polymer backbone and those on the methylene groups connecting the lateral chains to the polymer backbone), 3.99 (2H, protons on the carbon adjacent to the double bond), 5.20 (2H, vinylic), 5.88 (1H, vinylic). ¹³C NMR (400 MHz, DMSO-d₆, ppm, for n=5): 13.21, 18.17, 23.49, 27.07, 28.29, 29.43, 31.29, 32.78, 35.27, 36.10, 42.22, 46.59, 47, 15, 48.01, 69.99, 70.68, 72.13, 79.11, 116.97, 126.79, 128.59, 133.88, 136.08.

### Preparation of CA-OH(AGEₙ-COOH)₄ with Carboxylic Acid Pendant Groups

The addition of 3-mercaptopropionic acid on the CA-OH(AGEₙ)₄ was performed by dissolving 1 g of the polymer in 2.75 mL THF. Then, 5 and 0.15 equivalents, according to the number of double bonds, of 3-mercaptopropionic acid and AIBN were added successively and the solution was refluxed for 5 hours. The most volatile compounds were removed with a rotary evaporator and a vacuum distillation was performed to remove the less volatile ones. The viscous liquid resulted from this distillation was purified by dialysis. Yield: >94%, efficiency of the subsequent addition of thiolated compounds: 85% to 95%, see Table 2. ¹H NMR (400 MHz, acetone-d₆); 0.74 (18-CH₃), 1.87 (4H, protons on the β carbon to the oxygen atom on the lateral chain and those on the carbon adjacent to the carboxyl group), 2.65 and 2.80 (4H, protons on the two carbons adjacent to the sulfur atom), 3.59 (5H, protons on the polymer backbone and those on the methylene groups connecting the lateral chains to the polymer backbone). ¹³C NMR (400 MHz, DMSO-d₆, ppm, for n=5): 27.31, 28.68, 30.24, 35.47, 52.28, 69.70, 79.04, 173.93.

### Preparation of CA-OH(AGEₙ-NH₂)₄ with Amino Pendant Groups

CA-OH(AGEₙ)₄ was dissolved in methanol ( 1/20, w/v), 5 equivalents of cysteamine hydrochloride and 0.5 equivalent of AIBN per double bond were added. After refluxing the solution for 36 hours, methanol was evaporated and the residual syrup was dissolved in chloroform. Sodium hydroxide pellets were added to neutralize the ammonium chloride salt and the organic layer was washed one time with water. After solvent evaporation, the resulting viscous liquid was purified by dialysis. Yield: >95%, efficiency of the subsequent addition of thiolated compounds: 80% to 102%, see Table 2. ¹H NMR (400 MHz, CDCl₃, n=5): 0.70 (18-CH₃), 1.86 (protons on the β carbon to the oxygen atom on the lateral chains), 2.08 (protons on the nitrogen atom), 2.63 (4H, protons on the two carbons adjacent to the sulfur atom), 2.90 (2H, protons on the carbon adjacent to the amino group), 3.55 (5H, protons on the polymer backbone and those on the methylene groups connecting the lateral chains to the polymer backbone). ¹³C NMR (400 MHz, methanol-d₄, ppm, for n=5): 23.43, 27.75, 28.24, 29.95, 34.50, 40.70, 69.91, 71.12, 79.19.

### Partial Acetylation of CA-OH(AGEₙ-NH₂)₄

CA-OH(AGE₅-NH₂)₄ was dissolved in dry dimethylformamide (1:2 w/v) and freshly distilled acetic anhydride was added dropwise. After 45 minutes, solid sodium hydroxide was added to the solution to neutralize the acidic medium. The solution was dialyzed against water and freeze-dried. Yield: 75% ¹H NMR (400 MHz, CDCl₃, n=5 with 5% acetylation): 0.68 (18-CH₃), 1.87 (protons on the β carbon to the oxygen atom on the lateral chains), 2.01 (CH₃-amide), 2.22 (protons on the nitrogen atom), 2.62 (4H, protons on the two carbons adjacent to the sulfur atom), 2.88 (2H, protons on the carbon adjacent to the amino group), 3.53 (5H, protons on the polymer backbone and those on the methylene groups connecting the lateral chains to the polymer backbone). ¹³C NMR (400 MHz, CDCl₃, ppm, for n=5 with 5% acetylation): 23.61, 28.81, 30.10, 36.54, 39.20, 41.44, 51.17, 70.14, 71.30, 79.17, 171.03.

### Characterization

In order to find the degree of thiolation onto the allylic functions, back titrations were performed. The sample (0.1 g) was first dissolved in 10 mL of 0.1 M standardized solution of HCl or NaOH for the amine and acid series, respectively, and then 20 mL of milli-Q water were added. The back titration with acid, for CA-OH(AGE₅-COOH)₄, or base, for CA-OH(AGE₅-NH₂)₄, was started and the pH was monitored in order to calculate the pKₐ, the first and the second equivalent points (Table 2).

Table 2 presents the molecular weight and polydispersity obtained by ¹H NMR and SEC for the allylic CA-OH(AGEₙ)₄ polymers. The ¹H NMR results are in good accordance with the experimental feed ratios.

¹H NMR was used to confirm the star-shaped architecture of the polymers. The alcohol end groups were reacted with trifluoroacetic anhydride which shifted the adjacent protons to a different chemical shift (5.36 ppm,). The ratio of the integrations of these protons to the double bond protons yields the polymer arm length. The results indicate that all four positions (3, 7, 12, 24) have been initiated.

We used ¹H NMR, acid-base titration and elemental analysis to verify the completion of the addition of the thiol groups onto the double bonds. Both ¹H NMR and elemental analysis provided similar values. In the case of the titration, data obtained still agree with the two previous methods for the amines CA-OH(AGEₙ-NH₂)₄, but not for the acids CA-OH(AGEₐ-COOH)₄. The lower degree of substitution determined by titration reflects the difficulty to perform a complete titration.

### Aggregation Studies

These star polymers were designed to modify the aggregation properties of cholic acid, which aggregates around 8 mM when the carboxylic group is ionized (pKₐ 4.6 to 5.0) (S. Reis, C. G. Moutinho, C. Matos, B. de Castro, P. Gameiro and J. Lima, Anal. Biochem., 2004, 334, 117-126). The attachment of amino and carboxylic functions on cholic acid should change the range of pH where aggregation occurs and also the critical aggregation concentration (CAC) depending on the polymer chain length on the steroidal backbone.

Conductivity is a reliable and sensitive method to determine the critical micellar concentration (CMC). Conductivity experiments were performed with an Orion conductivity cell 018010 in a thermostated bath. The samples were prepared with standardized sodium hydroxide or hydrochloric acid solutions in milli-Q water to obtain the desired final concentration. The conductivity of the solution was measured after equilibration. The solution was then diluted to the next concentration with milli-Q water and the measurements were repeated. The results in Table 2 show that the CAC shift to a lower concentration as the polymer chain length increases due to the lipophilic nature of the uncharged polymeric units.

### Thermosensitivity

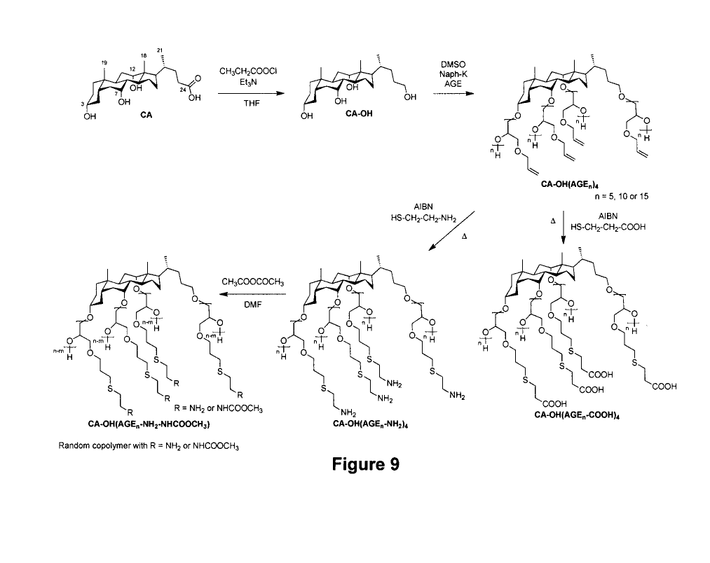

A simple addition of acetic anhydride to the amine set CA-OH(AGEₐ-NH₂)₄ results in the formation of the amide bonds on the amino groups to produce a statistical copolymer (see FIG. 9). The reaction of acetic anhydride and amino groups is almost quantitative, so that the experimental feed ratio should be the same proportion in the statistical copolymers formed.

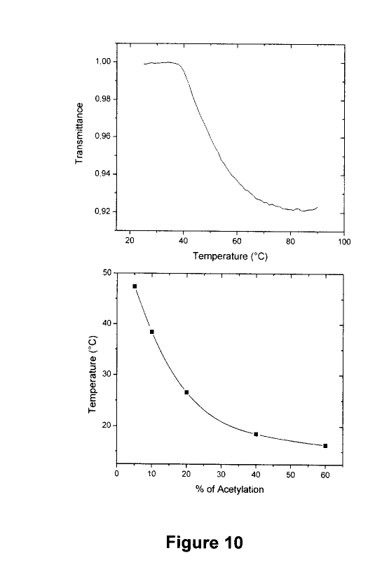

Cloud points (CP) were measured with a BioCary 300 UV-Vis spectrometer at 500 nm with solutions of the polymer in milli-Q water (0.1 wt %). Samples were successively heated and cooled at a rate of 0.1° C./min. CPs were taken as the onset of the heating cycles. Polymers with different degrees of acetylation, as evidenced by ¹H NMR, show different CPs. It is thus possible to tune the thermosensitivity by simply controlling the amount of acetic anhydride added to the polymers. The acetylation reaction is a simple reaction allowing an easy tuning of the CP. In this study, the polymers tested have CPs ranged from 16 to 53° C. (FIG. 10). This is a range of temperature of interest for biomedical applications.

TABLE 2
Addition of Thiolated Compounds onto the Synthesized
Polymers and their Aggregation.

| | | % of substitution by | | | | |
|---|---|---|---|---|---|---|
| | | | back | | | CAC |
| | | ¹H | titra- | elemental | | (× 10⁻³ |
| Compounds | n | NMR^{a} | tion^{b, c} | analysis^{d} | pKₐ | molale)^{e} |
| CA-OH | 5 | 93 | 89 ± 9 | 92 ± 1 | 5.0 | 17.5 ± 0.1 |
| (AGEₙ-COOH)₄ | | | 85 ± 7 | | | |
| | 10 | 100 | 94 ± 2 | 95.2 ± 0.3 | 5.4 | 12.8 ± 0.6 |
| | | | 87 ± 5 | | | |
| | 15 | 98 | 90 ± 5 | 94.8 ± 0.7 | 5.4 | 6.2 ± 0.6 |
| | | | 92 ± 5 | | | |
| CA-OH | 5 | 97 | 80 ± 3 | 101.6 ± 0.4 | 8.6 | 25.9 ± 0.1 |
| (AGEₙ-NH₂)₄ | | | 85 ± 2 | | | |
| | 10 | 98 | 93 ± 1 | 91 ± 1 | 8.5 | 17 ± 2 |
| | | | 86 ± 7 | | | |
| | 15 | 97 | 79 ± 5 | 89 ± 1 | 7.1 | 9 ± 2 |
| | | | 85 ± 3 | | | |

^{a}Error estimated to 7% for ¹H NMR
^{b}Initial average number of double bonds determined by ¹H NMR
^{c}The first value is calculated according to the first equivalent point and the second is calculated according to the volume difference between the two equivalent points
^{d}Based on the sulfur content
^{e}Measured at 25° C. and with 60% NaOH added for the acid series CA-OH(AGEₙ-COOH)₄ or 10% HCl added for the amine series CA-OH(AGEₙ-NH₂)₄

### Other Embodiments

From the foregoing description, it will be apparent to one of ordinary skill in the art that variations and modifications may be made to the invention described herein to adapt it to various usages and conditions. Such embodiments are also within the scope of the present invention.

All literature, patents, published patent applications cited herein are hereby incorporated by reference.

1. A polymer comprising:
(a) a cholane core comprising at least one derivatizable group covalently bonded thereto; and
(b) a hydrophilic polymer chain covalently bonded to the at least one derivatizable group.

2. The polymer of claim 1, wherein said cholane core is a bile acid.

3. The polymer of claim 2, wherein said bile acid is selected from the group consisting of cholic acid, chenodeoxycholic acid, glycocholic acid, taurocholic acid and deoxycholic acid.

4. The polymer of claim 1, wherein said at least one derivatizable group is selected from the group consisting of OH, NH₂, SH and CO₂H.

5. The polymer of claim 1, wherein said polymer comprises four hydrophilic polymer chain covalently bonded to said derivatizable group.

6. The polymer of claim 1, wherein said hydrophilic polymer chain consist in repeating units of epoxy compounds based on an ethylene oxide structure.

7. (canceled)

8. (canceled)

9. The polymer of claim 1, wherein said hydrophilic polymer chain is a poly(ethylene glycol) chain or a poly (allyl glycidyl ether) chain.

10. (canceled)

11. The polymer of claim 1, wherein said cholane core is a cholic acid having four derivatizable groups and said hydrophilic polymer chain is a poly(ethylene glycol) chain

12. (canceled)

13. A micellar aggregate comprising, in aqueous solution, a plurality of polymers, the polymers being different or the same, and being selected from the polymers as set forth claim 1.

14. A process for preparing an amphiphilic polymer, said process comprising:
a) in an aprotic solvent, partially deprotonating at least one derivatizable group of a cholane core to produce at least one deprotonated group; and
b) reacting the at least one deprotonated group with at least one hydrophilic polymer chain, the number of deprotonated group being equal to the number of hydrophilic chain, thereby forming an amphiphilic polymer.

15. A process for preparing the polymer as set forth in claim 11, said process comprising:
a) in an aprotic solvent, deprotonating four of the available hydroxyl groups of cholic acid to produce alkoxide groups; and
b) reacting said alkoxide groups with a sufficient amount of hydrophilic polymer chain to allow the formation of said polymer.

16. A polymer comprising:
a) a cholane core comprising at least one derivatizable group covalently bonded thereto;
b) a first monomer chain bonded to said at least one derivatizable group, wherein said first monomer chain optionally includes a first functional group adapted to be chemically modified; and
c) a second functional group located at the end of said first monomer chain.

17. (canceled)

18. The polymer of claim 16, further comprising a second monomer chain bonded to said first monomer chain, wherein said second monomer chain optionally includes a functional group adapted to be chemically modified or further comprising a second monomer chain comprising at least one unit bonded on each unit of said first monomer chain, said at least one unit of said second monomer optionally including a functional group adapted to be chemically modified.

19. (canceled)

20. (canceled)

21. The polymer of claim 16, wherein
said cholane core is a bile acid.

22. The polymer of claim 21, wherein said bile acid is selected from the group consisting of cholic acid, chenodeoxycholic acid, glycocholic acid, taurocholic acid and deoxycholic acid.

23. (canceled)

24. The polymer of claim 16, wherein said first monomer chain is poly(ethylene glycol).

25. The polymer of claim 18, wherein said second monomer chain is —CH₂—CHR—O—, wherein R is selected from the group consisting of H, alkyl, and R′X,
R′ being an alkyl chain and X being selected from the group consisting of a halide, SH, OH, COOH, NH₂ and OCH₂CH═CH₂.

26. (canceled)

27. The polymer of claim 16, wherein said second functional group is selected from the group consisting of transferring, asialoglycoprotein, antibodies, antibody factors, erythropoietin, epidermal growth factor (EGF), insulin, asialoorosomucoid, mannose-6-phosphate, mannose, Lewis X and sialyl Lewis X, N-acetyllactosamine, galactose, lactose, thrombomodiulin, fusogenic agents, polymisin B, hemagglutinin HA2, lysomotrophic agents, peptide, folic acid and nucleus localization signals (NLS).

28. The polymer of claim 16, wherein said second functional group is further modified to allow addition thereto of at least one monomer unit.

29. The polypeptide of claim 16, wherein one or both said first and second monomer chain are selected from the group consisting of methyl, ethyl, halomethyl, haloethyl, allyl, vinyl, protected hydroxymethyl and hydroxyethyl, protected aminoethyl and aminoethyl.
